(19) United States
(12) Patent Application Publication
(10) Pub. No.: US 2024/0148960 A1
(43) Pub. Date: May 9, 2024

(54) SURGICAL VACUUM DEVICE

(71) Applicant: LSI Solutions, Inc., Victor, NY (US)

(72) Inventor: Jude S. Sauer, Pittsford, NY (US)

(21) Appl. No.: 18/386,550

(22) Filed: Nov. 2, 2023

Related U.S. Application Data
(60) Provisional application No. 63/424,575, filed on Nov. 11, 2022.
(60) Provisional application No. 63/422,203, filed on Nov. 3, 2022.

(51) Int. Cl. A61M 1/00

(52) U.S. Cl. CPC A61M 1/86; A61M 1/7411; A61M 2039/1077

(57) ABSTRACT

A surgical device includes a handle portion having a first port and a second portion, and an elongated shaft is rotatably coupled to the handle portion. A first suction tip is removably secured to a distal end of the shaft, the first suction tip having a first cross-sectional shape that is configured to engage a first type of tissue on a patient. When the first port is coupled to the vacuum source, and when the second port is in a closed position, fluid at the first suction tip is passed through the shaft, into a fluid chamber of the handle portion, and out of the first port. When the first port is coupled to the vacuum source, and when the second port is in an open position, the fluid chamber is vented to the atmosphere, and fluid at the first suction tip is not displaced to the first port.

### RELATED APPLICATIONS

This patent application claims priority to U.S. Provisional Patent Application No. 63/422,203, filed Nov. 3, 2022, and U.S. Provisional Patent Application No. 63/424,575, filed Nov. 11, 2022, each of which is incorporated by reference herein in its entirety.

### FIELD OF THE INVENTION

The claimed invention relates to surgical devices, and more specifically to a vacuum device used in a surgical procedure.

### BACKGROUND

During a surgical procedure, blood and body fluids typically collect at the surgical site and obscure a surgeon's view of the surgical area. To clear the fluid from the treatment area and improve the surgeon's view, a vacuum aspiration system can be used. The vacuum aspiration system typically includes an aspiration cannula that is coupled to a vacuum source by a suction tube. The surgeon positions a distal tip of the aspiration cannula at the treatment site, and the fluid is sucked through the distal tip of the aspiration cannula and away from the treatment area due to negative pressure provided by the vacuum source. However, the distal tip of the aspiration cannula is typically an integrally-formed end portion of the aspiration cannula, and such a distal tip provides a uniform level of suction. Accordingly, when the distal tip of the aspiration cannula is positioned on or near tissue during aspiration, the tissue may be forcibly displaced towards the tip and into contact with the tip, potentially damaging the tissue. Further, when dealing with layers of delicate tissue, such as in minimally-invasive cardiac procedures, both proximal and distal layers of tissue may be engaged and displaced by the distal tip of the aspiration cannula.

Accordingly, there is a need for an aspiration device that can regulate the amount of suction force provided on a desired layer of delicate tissue to minimize trauma during aspiration, as well as to manipulate a proximal layer of tissue without disturbing a distal layer of tissue. Such single-layer manipulation allows for suturing of the proximal layer without inadvertently suturing the distal tissue in minimally-invasive suturing procedures.

### BRIEF DESCRIPTION OF THE DRAWINGS

FIG. 1 is a perspective view of an embodiment of a surgical device;

FIG. 2 is an exploded perspective view of the embodiment of the surgical device of FIG. 1;

FIG. 3 is a bottom view of the embodiment of the surgical device of FIG. 1;

FIG. 4 is a rear view of the embodiment of the surgical device of FIG. 1;

FIG. 5 is a front view of the embodiment of the surgical device of FIG. 1;

FIG. 6 is a side view of the embodiment of the surgical device of FIG. 1;

FIG. 7 is a cross-sectional view of the embodiment of the surgical device of FIG. 1 taken along section line 7-7 of FIG. 5;

FIG. 8 is a detailed view of a portion of the cross-sectional view of FIG. 7;

FIG. 9 is a cross-sectional view of the embodiment of the surgical device of FIG. 1 taken along section line 9-9 of FIG. 6;

FIG. 10 is a partial cross-sectional view of a portion of the handle portion showing the second port in a second closed position;

FIG. 11A is a partial view of an embodiment of a first suction tip secured to a distal end of a shaft of an embodiment of a surgical device;

FIG. 11B is a cross-sectional view of the embodiment of the first suction tip of FIG. 11A taken along section line 11B-11B of FIG. 11A;

FIG. 12A is a partial view of an embodiment of a second suction tip secured to a distal end of a shaft of an embodiment of a surgical device;

FIG. 12B is a cross-sectional view of the embodiment of the second suction tip of FIG. 11A taken along section line 12B-12B of FIG. 12A;

FIG. 13 is a cross-sectional view of the embodiment of a rotation adapter;

FIGS. 14A to 14H are various views of an additional embodiment of a suction tip configured to be removably secure to a distal end of a shaft of an embodiment of a surgical device;

FIGS. 15A to 15F are various views of a further embodiment of a suction tip configured to be removably secure to a distal end of a shaft of an embodiment of a surgical device; and

FIGS. 16A to 16H are various views of another embodiment of a suction tip configured to be removably secure to a distal end of a shaft of an embodiment of a surgical device.

### DETAILED DESCRIPTION OF THE INVENTION

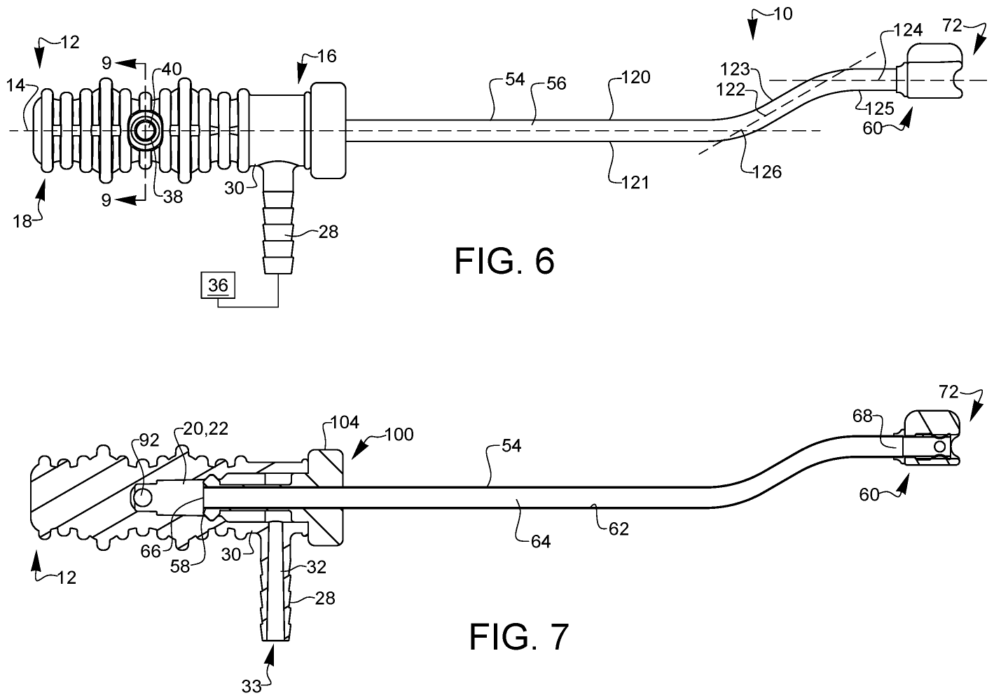

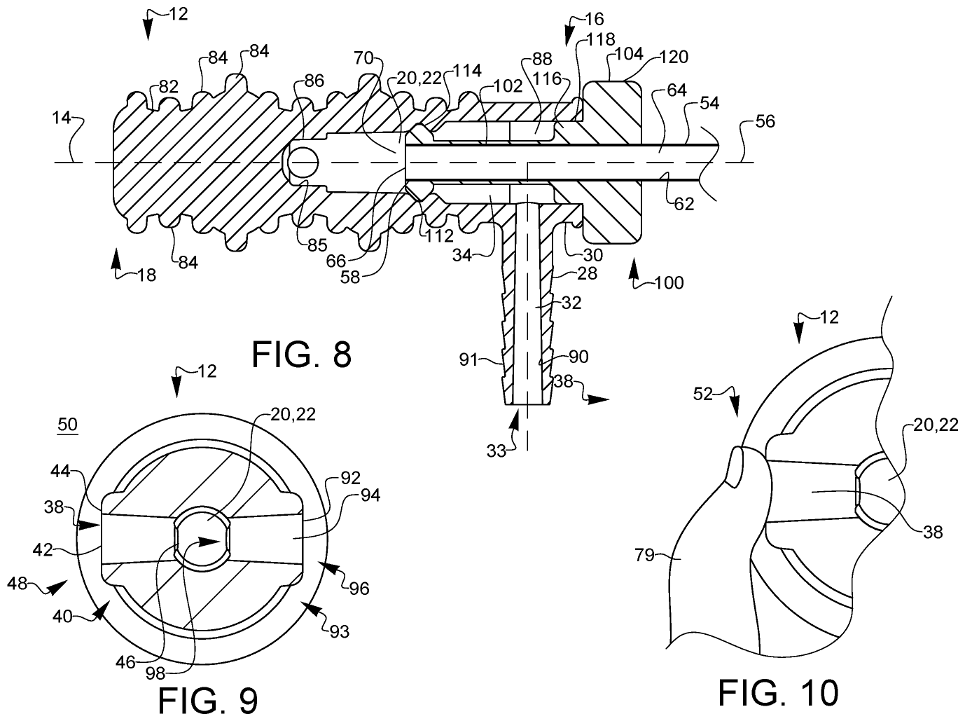

Referring to FIG. 6, an embodiment of a surgical device 10 includes a handle portion 12 extending along a handle axis 14 from a distal end 16 to a proximal end 18. With reference to the cross-sectional view of FIG. 8, the handle portion 12 also includes a fluid chamber 20 that extends within an interior portion 22 of the handle portion 12. The surgical device 10 further includes a first port 28 disposed on a first portion 30 of the handle portion 12, the first port 28 defining a first port channel 32 that extends from an open end portion 33 of the first port 28 to a first portion 34 of the fluid chamber 20 of the handle portion 12 such that the first port channel 32 is in fluid communication with the fluid chamber 20 of the handle portion 12. As illustrated in FIG. 6, the first port 28 is configured to be coupled to a vacuum source 36.

The surgical device 10 further includes a second port 38 disposed on a second portion 40 of the handle portion 12. As illustrated in the cross-sectional view of FIG. 9, the second port 38 defines a second port channel 42 that extends from an open end portion 44 of the second port 38 to a second portion 46 of the fluid chamber 20 of the handle portion 12 such that the second port channel 42 is in fluid communication with the fluid chamber 20 of the handle portion 12. When the second port 38 is in a first open position 48 (illustrated in FIG. 9), the fluid chamber 20 of the handle portion 12 is vented to the atmosphere 50. When the second port 38 is in a second closed position 52 (such as when a portion of a user's finger 79 covers the open end portion 44 of the second port 38, as illustrated in FIG. 10), the fluid chamber 20 of the handle portion 12 is not vented to the atmosphere 50.

Referring to FIG. 6, the surgical device 10 further includes a shaft 54 extending along a shaft axis 56 from a proximal end 58 (see FIG. 8) to a distal end 60. As shown in FIG. 7, one or more inner surfaces 62 of the shaft 54 defines a shaft channel 64 that extends from a shaft channel proximal end 66 at the proximal end 58 of the shaft 54 to a shaft channel distal end 68 at the distal end 60 of the shaft 54. With reference to FIG. 8, the shaft 54 is rotatably coupled to the handle portion 12 such that the shaft channel proximal end 66 is disposed within a third portion 70 of the fluid chamber 20 of the handle portion 12 such that the shaft channel 64 is in fluid communication with the fluid chamber 20 of the handle portion 12.

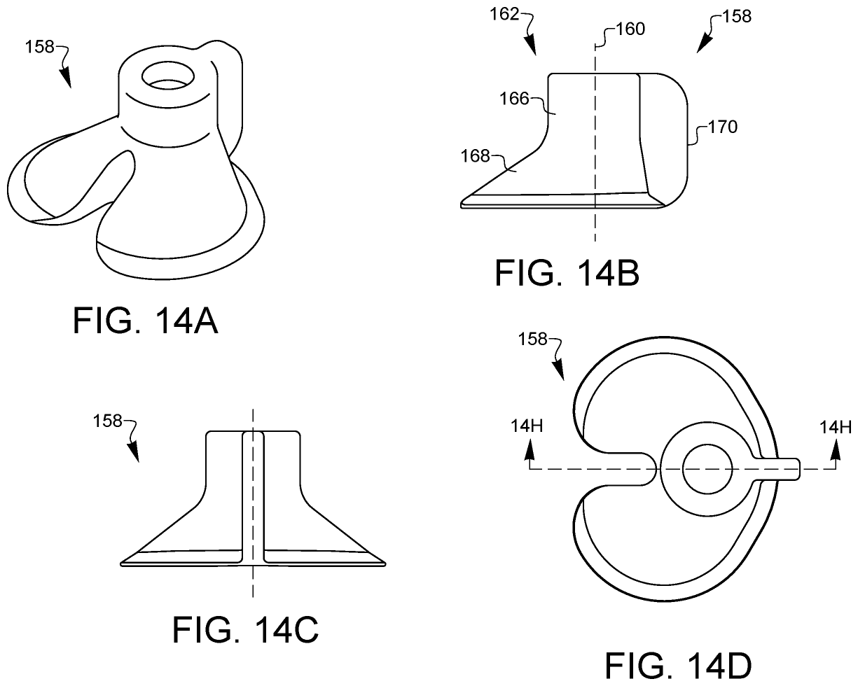

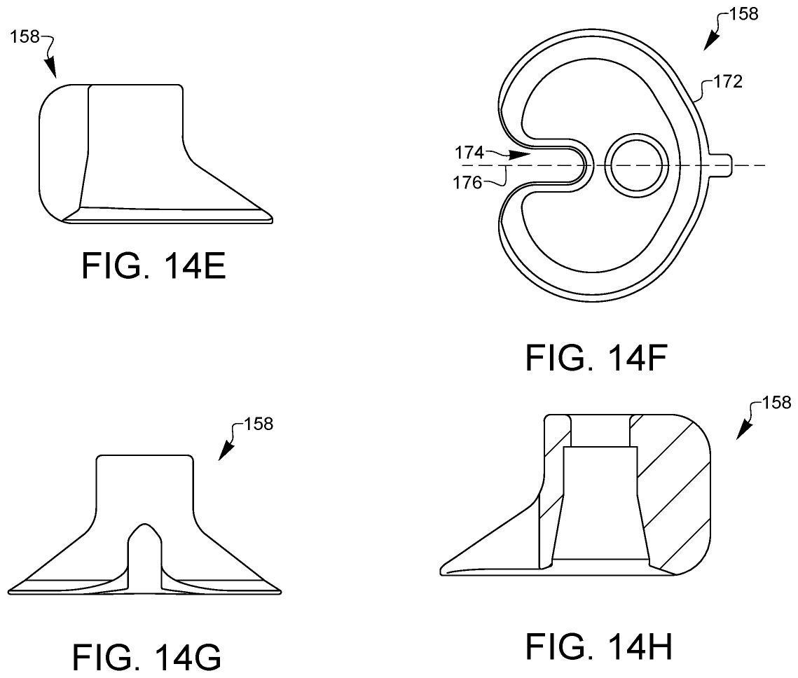

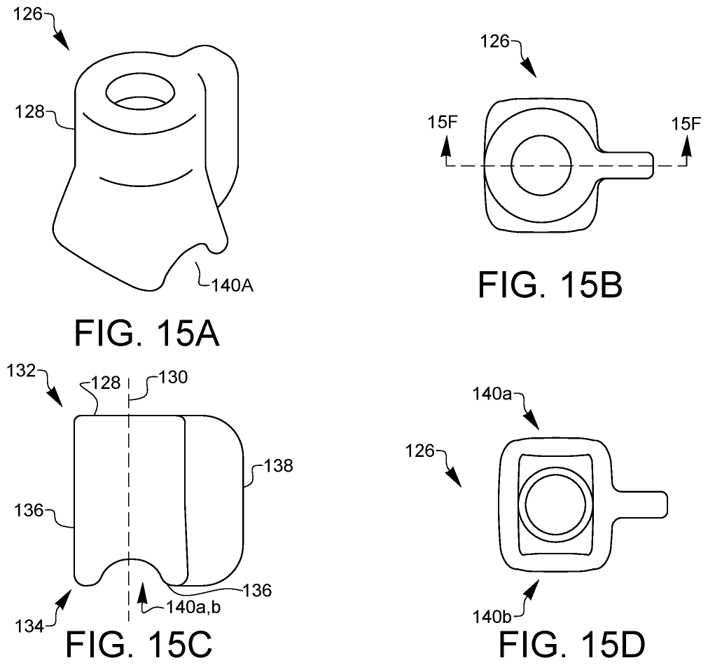

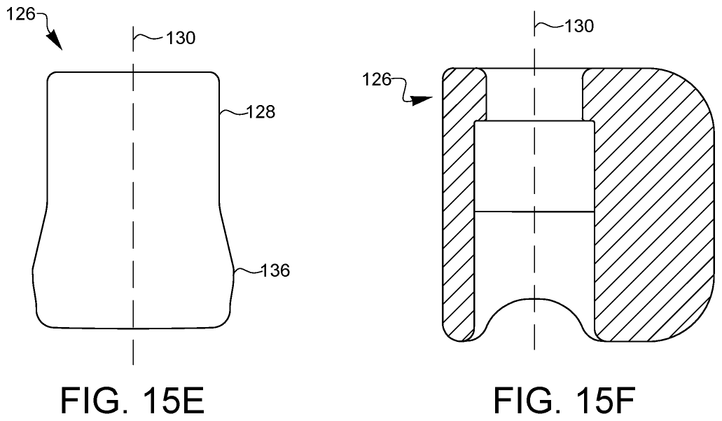

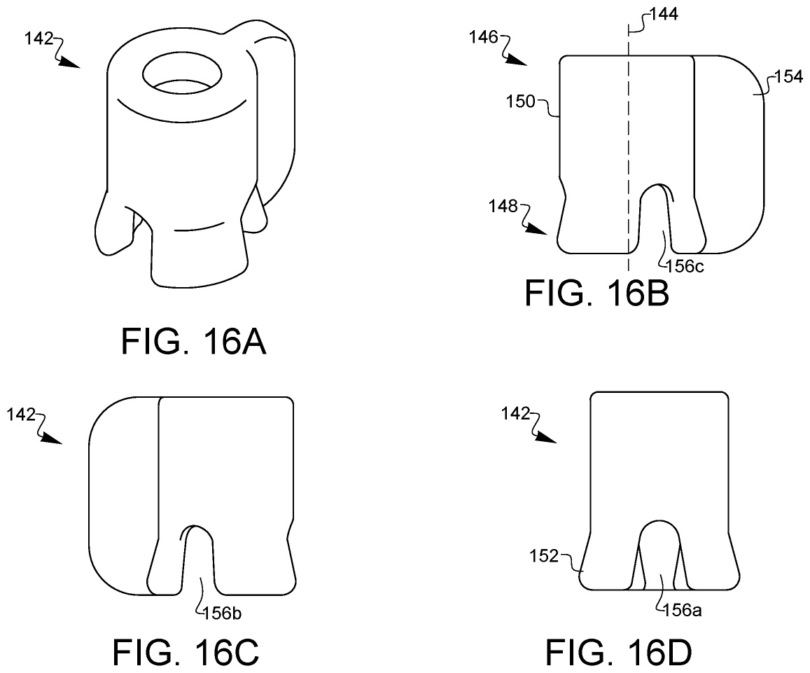

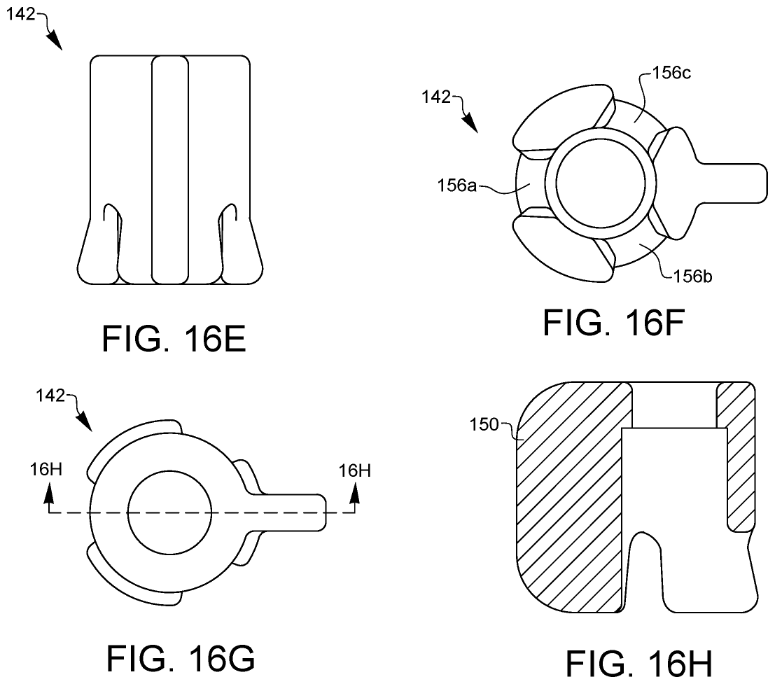

Turning again to FIG. 6, the surgical device 10 further includes a first suction tip 72 removably secured to the distal end 60 of the shaft 54. The first suction tip 72 has a first cross-sectional shape that is configured to engage a first type of tissue on a patient, wherein the first suction tip 72 comprises a deformable material. In some embodiments, the surgical device 10 may be part of a kit that includes the first suction tip 72 removably secured to the distal end 60 of the shaft 54. The kit may also include any number of suction tips having sizes, shapes, and/or material properties that may differ from the first suction tip 72. For example, the kit may include one or more of a second suction tip 80 (see FIG. 12A), a third suction tip 126 (see FIGS. 15A to 15F), a fourth suction tip 142 (see FIGS. 16A to 16H), and a fifth suction tip 158 (see FIGS. 14A to 14H), and each of these suction tips will be described in more detail in following sections.

With the first suction tip 72 secured to the distal end 60 of the shaft 54, and with the first port 28 coupled to the vacuum source 36 (see FIG. 6), and when the second port 38 is in the second closed position 52 (see FIG. 10), the distal end 60 of the shaft 54 is at a low pressure via the vacuum source 36 such that a portion of the first suction tip 72 engages a portion of the first type of tissue of the patient and body fluid at or adjacent to the portion of the first type of tissue of the patient displaces from the shaft channel distal end 68 through the shaft channel 64, into the fluid chamber 20, through the first port 28 channel, and out of the open end portion 33 of the first port 28.

In addition, when the first port 28 is coupled to the vacuum source 36, and when the second port 38 is in the first open position 48 (illustrated in FIG. 9), the fluid chamber 20 is vented to the atmosphere such that the distal end 60 of the shaft 54 is not at the low pressure of the vacuum source 36, and body fluid at or adjacent to the portion of the first type of tissue of the patient is not displaced from the shaft channel distal end 68 through the shaft channel 64.

So configured, the surgical device 10 provides a compact and ergonomic hand-held vacuum unit for removing fluids obscuring the surgeon's view of a treatment area during a surgical procedure, especially a minimally-invasive surgical procedure. The surgeon may easily position the shaft 54 in a desired rotational position and place the atraumatic first suction tip 72 at or adjacent to a treatment area to remove fluid from the treatment area. The surgical device 10 also provides a versatile tool for working with a variety of tissue types during a procedure. In particular, the first suction tip 72 having a first set of shapes/material properties optimized for use on a first tissue type (e.g., tissue having a first thickness and/or texture) may be swapped with the second suction tip 80 (see FIG. 12A) having a second set of shapes/material properties optimized for use on a second tissue type (e.g., tissue having a second thickness and/or texture). In some embodiments, a desired tip may be selected to lift a proximal layer of a certain type of tissue from an adjacent distal layer of tissue, and this separation may allow the proximal tissue layer to be sutured without inadvertently also suturing the distal tissue layer. The first suction tip 72 may also have different features than the second section tip 80, such as slots or differently shaped edges, that achieve different suction levels or that can accommodate different types of fluids having different viscosities or particulate levels. Such features may also accommodate surface features of the tissue to maintain adequate suction levels.

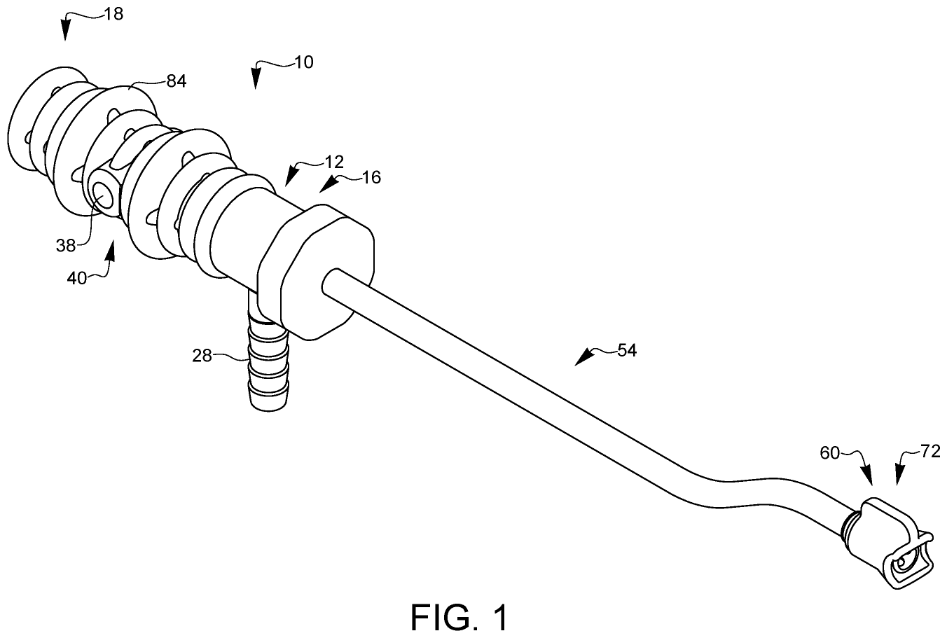

Turning to the surgical device 10 in more detail, FIGS. 1, 3, and 6 illustrate the handle portion 12 that extends along the handle axis 14 from a distal end 16 to a proximal end 18, and the handle portion 12 may be generally shaped to grasped by a hand of the user. In particular, as illustrated in FIG. 8, the handle portion 12 may be partially defined by a contoured (e.g., non-cylindrical or partially non-cylindrical) outer surface 82 that generally extends along the handle axis 14. A plurality of gripping members 84 may radially extend from the outer surface 82, and each of the gripping members 84 may have the shape of a disk that is normal to the handle axis 14 with a center that intersects the handle axis 14. Each of the gripping members 84 may be uniformly spaced from adjacent gripping members 84, but the diameter of adjacent gripping members 84 may be different. In some embodiments, the diameter of adjacent gripping members 84 is sized such that the gripping members 84 extends a desired distance (such as a uniform or substantially uniform distance) from corresponding adjacent portions of the outer surface 82.

Referring to FIG. 8, the handle portion 12 may also include one or more interior surfaces 85 that cooperate to define the interior portion 22 of the handle portion 12, and the one or more of the one or more interior surfaces 85 may define the fluid chamber 20 that extends within (or is a portion of) the interior portion 22 of the handle portion 12. The fluid chamber 20 (and the one or more interior surfaces 85 that define the fluid chamber 20) may have any suitable shape (or combination of shapes), such as an elongated shape that generally extends along the handle axis 14 from a proximal end 86 to a distal end 88.

With reference to FIGS. 1, 3, and 6, the surgical device 10 further includes the first port 28 disposed on the first portion 30 of the handle portion 12, and the first port 28 may be a stem or other projection that is integrally formed with the handle portion 12 such that the first port 28 may be a portion of the handle portion 12. In some embodiments, the first port 28 may include one or more separate parts that are coupled to the handle portion 12. As illustrated in the cross-sectional view of FIG. 8, one or more inner surfaces 90 of the first port 28 may define the first port channel 32 that extends from an open end portion 33 of the first port 28 (at a first end of the first port 28) to a second end of the first port channel 32 at or adjacent to the first portion 34 of the fluid chamber 20 of the handle portion 12. So configured, the first port channel 32 is in fluid communication with the fluid chamber 20 of the handle portion 12.

All or a portion of an exterior surface 91 of the first port 28 may be configured to mate with or couple to a tube or other conduit (not shown) that may itself be connected to or be in fluid communication with one or more suction sources, such as the vacuum source 36 illustrated in FIG. 6. For example, the exterior surface 91 of the first port 28 may include a plurality of ridges that may be configured to engage an interior surface of a tube (not shown) that is in communication with the communication with the vacuum source 36. In other embodiments, all or a portion of the exterior surface 91 of the first port 28 may include a plurality of threads or any other coupling mechanism or combination of coupling mechanisms.

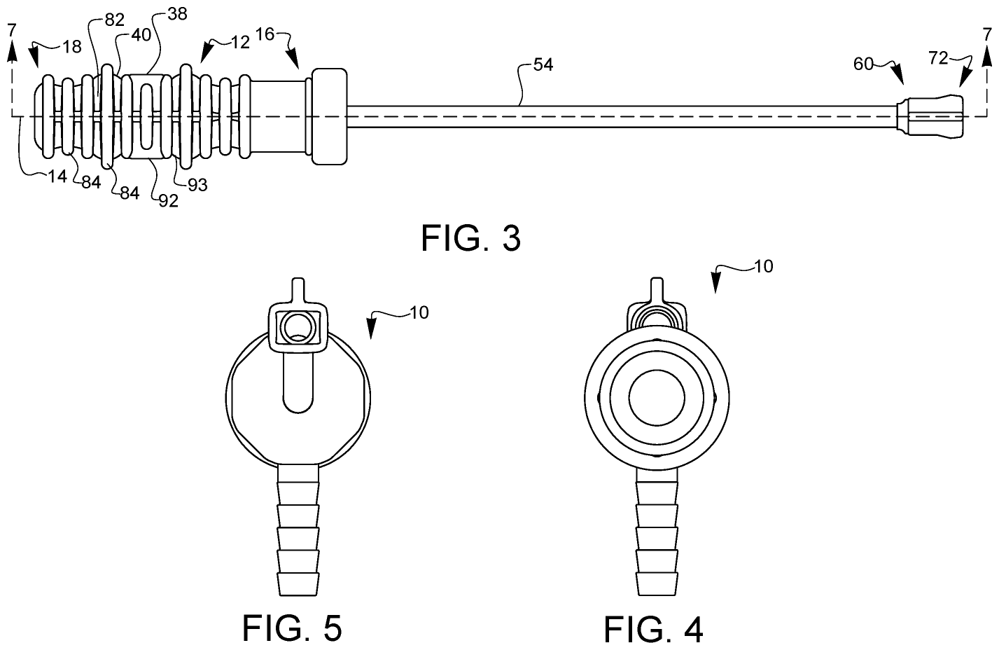

With reference to FIGS. 1, 2, 3, and 5, the surgical device 10 further includes the second port 38 disposed on, at, or adjacent to the second portion 40 of the handle portion 12. As illustrated in the cross-sectional view of FIG. 9, the second port 38 define a second port channel 42 that extends from the open end portion 44 of the second port 38 (at a first end of the second port 38) to a second end of the second port channel 42 at or adjacent to the second portion 46 of the fluid chamber 20 of the handle portion 12. The second port channel 42 may be an aperture through the handle portion 12 at or adjacent to the second portion 40 of the handle portion 12. So configured, the second port channel 42 is in fluid communication with the fluid chamber 20 of the handle portion 12. Thus, when the second port 38 is in a first open position 48 (illustrated in FIG. 9), the fluid chamber 20 of the handle portion 12 is vented to the atmosphere 50, and when the second port 38 is in a second closed position 52, the fluid chamber 20 of the handle portion 12 is not vented to the atmosphere 50. By manually controlling the second port 38, a surgeon (or other user) may control the flow of fluid from the treatment area through the first port channel 32 due to the vacuum source 36. In particular, when the surgeon desires to maintain or create suction at the treatment area, the surgeon may cover the open end portion 44 of the second port 38 with a portion of a surgeon's finger 79, such as the index finger, as illustrated in FIG. 10. The index finger 79 may then be removed from the open end portion 44 of the second port 38 to disrupt the suction at the treatment area. One having ordinary skill in the art would recognize that any other conduits in fluid communication with both the atmosphere 50 and the fluid chamber 20 of the handle portion 12 (such as a third port 92, which is illustrated in FIGS. 3 and 9 and will be described in more detail below) must also be closed or blocked when the second port 38 is closed or blocked to create suction at the treatment area.

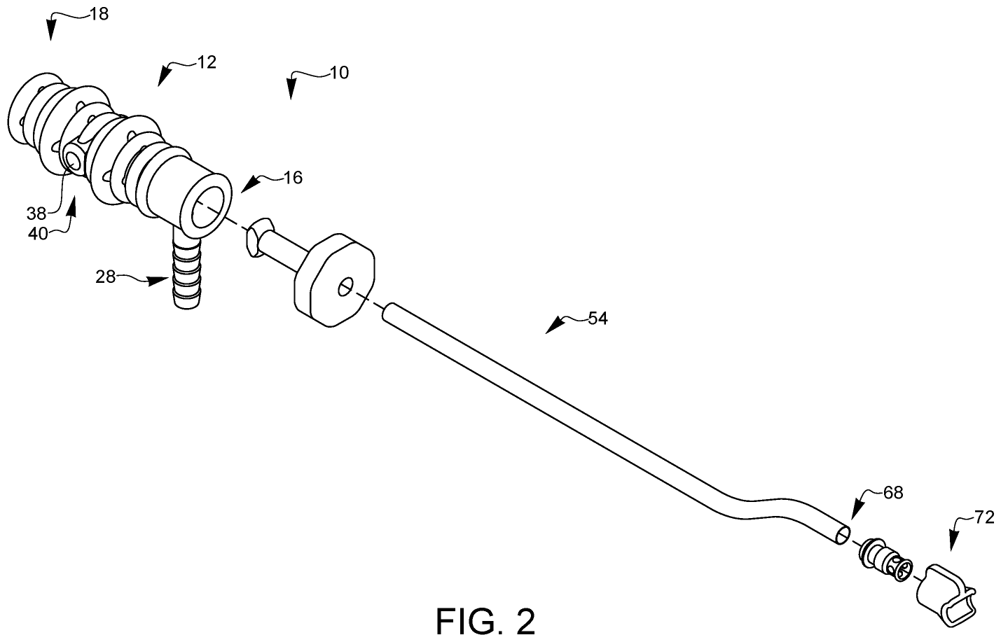

With reference to FIGS. 2 and 9, the surgical device 10 may also include the third port 92 disposed on, at, or adjacent to a third portion 93 of the handle portion 12. As illustrated in the cross-sectional view of FIG. 9, the third port 92 may be identical to, and axially aligned with, the second port 38 on the handle portion 12. In particular, the third port 92 may define a third port channel 94 that extends from an open end portion 96 of the third port 92 (at a first end of the third port 92) to a second end of the third port channel 94 at or adjacent to a portion 98 of the fluid chamber 20 of the handle portion 12. The third port channel 94 may be an aperture through the handle portion 12 at or adjacent to the third portion 93 of the handle portion 12. So configured, the third port channel 94 is in fluid communication with the fluid chamber 20 of the handle portion 12. Thus, when the third port 92 is in a first open position (illustrated in FIG. 9), the fluid chamber 20 of the handle portion 12 is vented to the atmosphere 50, and when the third port 92 is in a second closed position (not shown, but identical to the second closed position 52 of the second port 38 illustrated in FIG. 10), the fluid chamber 20 of the handle portion 12 is not vented to the atmosphere 50. The third port 92 may be controlled identically to the second port 38, and when the third port 92 and the second port 38 are both closed (e.g., each by a surgeon's finger), fluid may flow from the treatment area through the first port channel 32 due to the vacuum source 36. However, when one or both of the third port 92 or the second port 38 are in the open position, the suction may be disrupted and no fluid is removed from the treatment area. In some embodiments, the handle portion 12 of the surgical device 10 only includes the second port 38 and does not include the third port 92.

In other embodiments, the second port 38 and/or the third port 92 may be selectively closed or blocked in any suitable manner, such as by a removable plug, a valve, etc. In other embodiments, the second port 38 and/or the third port 92 may not each be an aperture formed in the handle portion 12, but may be any suitable structure that is coupled to the handle portion 12.

Configured as described, the handle portion 12, the first port 28, the second port 38, and the third port 92 may be a unitary part integrally formed as a single part, and the material may be a plastic, injection molded part. The part may be plastic, and may be, for example, ABS or a deformable polymer material. However, the handle portion 12, the first port 28, the second port 38, and the third port 92 may be an assembly of two or more parts that are coupled to form the handle portion 12.

Turning to FIGS. 1 to 3 and 6 to 8, the surgical device 10 may further include a rotational adapter 100 fixedly secured to a portion of the shaft 54 at or adjacent to the proximal end 58 of the shaft 54. As illustrated in the cross-sectional view of FIG. 13 that is a detail of FIG. 8, the rotation adapter 100 includes an insertion portion 102 and an engagement portion 104, and the insertion portion 102 is elongated and extends along an adapter axis 105 from a first end 106 at a proximal end 108 of the rotational adapter 100 to a second end 109 at a proximal end 110 of the engagement portion 104. The insertion portion 102 may be cylindrical and may be adapted to receive a portion of the shaft 54 such that the shaft axis 56 is aligned with the adapter axis 105. The portion of the shaft 54 is fixed or secured to the insertion portion 102 and rotates with the insertion portion 102 when a user rotates the engagement portion 104 of the rotational adapter 100. A projection 112 is disposed at or adjacent to the first end 106 of the insertion portion 102, and the projection 112 is adapted to be received and rotate within a corresponding annular recess 114 (see FIG. 8) formed in the interior portion 22 of the handle portion 12. The projection 112 is shaped, when viewed along the adapter axis 105, to have a generally rectangular shape (having opposing linear edges, as illustrated in FIG. 2) that is configured to allow flow around the projection 112 within the fluid chamber 20 so as to not block or completely obstruct flow around the projection 112 within the fluid chamber 20. A sealing projection 116 at or adjacent to the second end 109 of the insertion portion may be cylindrical and may be configured to be received within a corresponding cylindrical recess 118 (see FIG. 8) in the interior portion 22 of the handle portion 12. The cylindrical recess 118 may be disposed at or adjacent to the open distal end 16 of the handle portion 12. The sealing projection 116 may rotate within the cylindrical recess 118 and seal the distal end 88 of the fluid chamber 20.

Referring again to FIG. 13, the rotation adapter 100 includes the engagement portion 104. The proximal end 110 of the engagement portion 104 is disposed at the second end 109 of the insertion portion 102 and a distal end 117 of the engagement portion 104 is disposed at a distal end 119 of the rotation adapter 100. The engagement portion 104 may have the shape of a disc, and a diameter of the disc may be greater than an outer diameter of the distal end 16 of the handle portion 12 such that the engagement portion 104 radially extends beyond the distal end 16 of the handle portion 12. The engagement portion 104 may have a circumferential portion 120 disposed around or along the circumference of the disc, and the circumferential portion 120 may be adapted or configured to be rotated by a surgeon or other user to rotate the rotation adapter 10 and the shaft 54 about the shaft axis 56 (or about the adapter axis 105). For example, the circumferential portion 120 may have a polygonal profile or a circular profile when viewed along the adapter axis 105.

With reference to FIGS. 1, 2, 3, 6, 7, and 8, the surgical device 10 further includes the shaft 54 that extends along the shaft axis 56 from a proximal end 58 (see FIG. 8) to a distal end 60. Referring to FIG. 7, one or more inner surfaces 62 of the shaft 54 defines the shaft channel 64 that extends from the shaft channel proximal end 66 at the proximal end 58 (see FIG. 8) of the shaft 54 to the shaft channel distal end 68 at the distal end 60 of the shaft 54. FIG. 8 illustrates that the shaft channel proximal end 66 may be disposed within the third portion 70 of the fluid chamber 20 of the handle portion 12 such that the shaft channel 64 is in fluid communication with the fluid chamber 20 of the handle portion 12.

The one or more inner surfaces 62 of the shaft 54 that define the shaft channel 64 may have any suitable shape or combination of shapes allow a fluid to flow through the shaft channel 64. For example, the one or more inner surfaces 62 may have a circular cross-sectional that may be uniform or generally uniform from the proximal end 58 of the shaft 54 to the distal end 60 of the shaft 54.

Referring to FIG. 6, all or a portion of the shaft axis 56 may be non-linear. In particular, the shaft axis 56 may include a first axis segment 120 corresponding to a first shaft segment 121, a second axis segment 122 corresponding to a second shaft segment 123, and a third axis segment 124 corresponding to a third shaft segment 125. The first shaft segment 121 may extend from a proximal end 58 of the shaft 54 to a distal end of the first shaft segment 121 that is a proximal end of the second shaft segment 123, and the first axis segment 120 may be coaxially aligned with (or parallel to) the handle axis 14. The second shaft segment 123 may extend from the proximal end at the distal end of the first shaft segment 121 to a distal end at a proximal end of the third shaft segment 125. The second axis segment 122 may be linear and may be disposed at an oblique angle (for example, and angel between 10 degrees and 45 degrees) relative to the first axis segment 120. However, the second axis segment 122 may be none-linear, and may be an arc, a segment of a circle, or any combination of linear and curved shapes. The third shaft segment 125 may extend from the proximal end at the distal end of the second shaft segment 123 to a distal end at the distal end 60 of the shaft 54. The third axis segment 124 may be linear and may be parallel to, and offset from, the first axis segment 120. However, in some embodiments, the shaft axis 56 may be linear from the proximal end 58 of the shaft 54 to the distal end 60 of the shaft 54, and the shaft axis 56 may be coaxially aligned with (or parallel to) the handle axis 14.

With reference to FIG. 6, the shaft 54 may be rotatably coupled to the handle portion 12 such that the shaft may rotate about the shaft axis 56 (or the first axis segment 120) of the shaft 54. As previously explained, a portion of the shaft 54 may be secured to or fixed to the rotational adapter 100, which may be rotatably coupled to the handle portion 12 such that when a user rotates the engagement portion 104 about the adapter axis 105, the rotational adapter 100 and the shaft rotate about the shaft axis 56 (or the first axis segment 120) of the shaft 54. This shape of the shaft 54 and the rotational coupling with the handle portion 12 allows a surgeon to precisely place the first suction tip 72 at the distal end 60 of the shaft 54 at a desired treatment area in the limited space associated with minimally-invasive surgery. In some embodiments, the shaft 54 may be fixedly secured to the handle portion 12 such that the shaft does not rotate about the shaft axis 56.

The shaft 54 may be manufactured using any suitable material or combination of materials. For example, the shaft 54 may be a metal tube that may be bent in a separate forming process. However, the shaft 54 may be an injection molded part, such as a one-piece unitary part that is folded about a living hinge to assemble the tube.

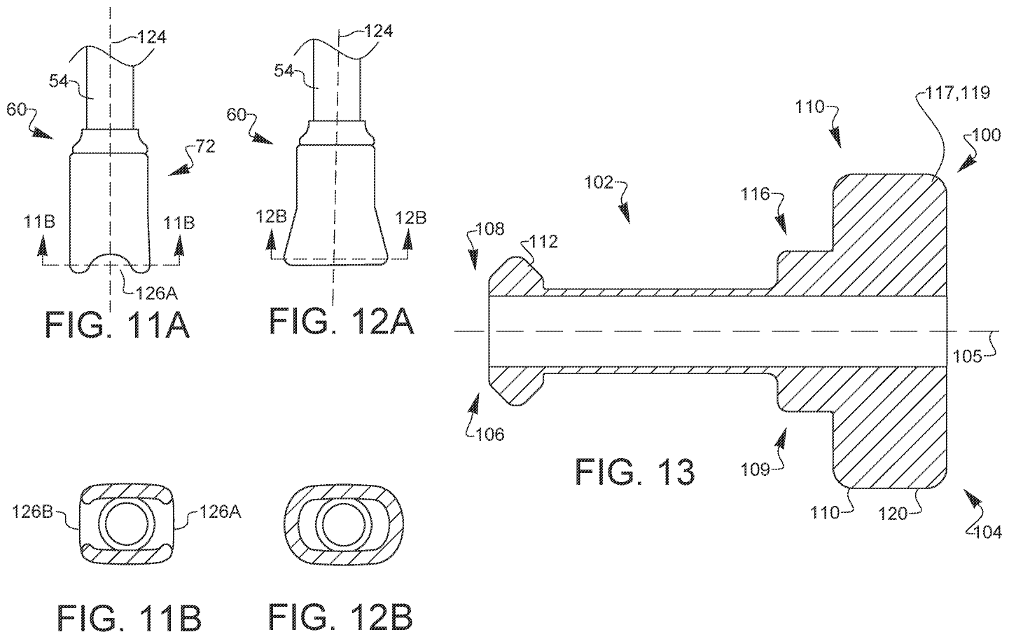

With reference to FIGS. 1, 2, 3, 6, and 7, the surgical device 10 further includes the first suction tip 72 removably secured to the distal end 60 of the shaft 54. The first suction tip 72 has a shape and/or material properties that are specifically configured to engage a first type of tissue on a patient during a procedure. For example, with reference to FIG. 11A, the first suction tip 72 may have a first cross-sectional shape taken along a plane normal to the third axis segment 124 (or the shaft axis 56) at a point adjacent to a distal end of the first suction tip 72, as illustrated in FIG. 11B. The first cross-sectional shape may include a rectangular or substantially rectangular side wall that defines an enclosed space that is configured to surround all or a portion of a treatment area. A pair of slots formed on opposing lateral walls may extend proximally from the distal end of the first suction tip 72, and the slots 126a, 126b may be provided such that relative thin tissue (or a relatively thin portion of tissue that is adjacent to a thicker tissue) may be forced into the slots 126a, 126b and into the enclosed space first suction tip 72 when the vacuum source 36 is engaged, thus separating the relatively thin portion of tissue from the thicker tissue. All or a portion of the first suction tip 72 comprises a deformable material, such as a polymer, such that the first suction tip 72 is atraumatic.

In some embodiments, the surgical device 10 may be part of a kit that includes the first suction tip 72 and the separate second suction tip 80 that can be secured to the distal end 60 of the shaft instead of the first suction tip 72. As previously explained, the second suction tip 80 may have a shape and/or material properties that are specifically configured to engage a second type of tissue on a patient during a procedure. For example, with reference to FIG. 12A, the second suction tip 80 may have a second cross-sectional shape taken along a plane normal to the third axis segment 124 (or the shaft axis 56) at a point adjacent to a distal end of the second suction tip 80, as illustrated in FIG. 12B. The second cross-sectional shape may include an oval-shaped side wall that defines an enclosed space that is configured to surround all or a portion of a treatment area. The second cross-sectional shape may be specifically configured to engage a second type of tissue that is different that the first type of tissue associated with the first suction tip 72. For example, the second suction tip 80 may be configured to engage relatively thick tissue and displace all or a portion of the relatively thick tissue during a procedure when the vacuum source 36 is engaged. All or a portion of the second suction tip 80 comprises a deformable material, such as a polymer, such that the second suction tip 80 is atraumatic.

Any number of additional suction tips (not shown) may be provided to be removably attached to the distal end of 60 the shaft 54, and each additional suction tip may have a unique shape and/or set of material properties configured to engage a specific type of tissue or optimally perform a specific task in a procedure.

FIGS. 15A to 15F illustrate a further embodiment of a third suction tip 126 that may be similar to the first suction tip 72. The third suction tip 126 may extend along an axis 130 from a proximal end 132 to a distal end 134, and the third suction tip 126 may have an upper wall portion 128 that extends along the axis 130 from the proximal end 132 towards the distal end 134 and has a substantially cylindrical shape. The third suction tip 126 may also have a lower wall portion 136 that extends along the axis 130 from the end of the upper wall portion 128 to the distal end 134, and the lower wall portion 136 may have a substantially rectangular shape. A tab portion 138 may extend from the proximal end 132 to the distal end 134. Like the first suction tip 72, the third suction tip 126 may include a pair of slots 140a, 140b.

FIGS. 16A to 16H illustrate a further embodiment of a fourth suction tip 142 that may be similar to the first suction tip 72 and third suction tip 126. The fourth suction tip 142 may extend along an axis 144 from a proximal end 146 to a distal end 148, and the fourth suction tip 142 may have an upper wall portion 150 that extends along the axis 144 from the proximal end 146 towards the distal end 148 and has a substantially cylindrical shape. The fourth suction tip 142 may also have a lower wall portion 152 that extends along the axis 144 from the end of the upper wall portion 150 to the distal end 148, and the lower wall portion 152 may have a substantially conical shape that flares outwardly as the lower wall portion 152 extends towards the distal end 148. A tab portion 154 may extend from the proximal end 146 to the distal end 148. The fourth suction tip 142 may include a plurality of slots 156a, 156b, 156c that each function similarly to the slots of the first suction tip 72. However, the slots 156a, 156b, 156c may be elongated and may have a length greater than its width at the slot 156a, 156b, 156c extends proximally from the distal end 148.

FIGS. 14A to 14H illustrate a further embodiment of a fifth suction tip 158. The fifth suction tip 158 may extend along an axis 160 from a proximal end 162 to a distal end 164, and the fifth suction tip 158 may have an upper wall portion 166 that extends along the axis 160 from the proximal end 162 towards the distal end 164 and has a substantially cylindrical shape. The fifth suction tip 158 may also have a lower wall portion 168 that extends along the axis 160 from the end of the upper wall portion 166 to the distal end 164, and the lower wall portion 168 may have a shape that flares outwardly as the lower wall portion 152 extends towards the distal end 148. A circumferential edge 172 (see FIG. 14F) at the distal end 164 of the lower wall portion 168 may have the shape of an oval or circle having an inwardly projecting recess 174 that extends along a reference axis 176 to give the circumferential edge 172 the general shape of a kidney or a bean. The reference axis 176 may be normal or substantially normal to the axis 160, and the circumferential edge 172 may be symmetrical about the reference axis 176. Such a shape defines a particularly shaped enclosed space that is configured engage a delicate tissue, such as aortic tissue. A tab portion 170 may extend from the proximal end 162 to the distal end 164. The fifth suction tip 158 may not have any slots.

Various advantages of a surgical device have been discussed above. Embodiments discussed herein have been described by way of example in this specification. It will be apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and the scope of the claimed invention. The drawings included herein are not necessarily drawn to scale. Additionally, the recited order of processing elements or sequences, or the use of numbers, letters, or other designations therefore, is not intended to limit the claims to any order, except as may be specified in the claims. Accordingly, the invention is limited only by the following claims and equivalents thereto.

What is claimed is:

1. A surgical device comprising:
a handle portion extending along a handle axis from a distal end to a proximal end, the handle portion having a fluid chamber that extends within an interior portion of the handle portion;
a first port disposed on a first portion of the handle portion, the first port defining a first port channel that extends from an open end portion of the first port to a first portion of the fluid chamber of the handle portion such that the first port channel is in fluid communication with the fluid chamber of the handle portion, wherein the first port is configured to be coupled to a vacuum source;
a second port disposed on a second portion of the handle portion, the second port defining a second port channel that extends from an open end portion of the second port to a second portion of the fluid chamber of the handle portion such that the second port channel is in fluid communication with the fluid chamber of the handle portion, wherein when the second port is in a first open position, the fluid chamber of the handle portion is vented to the atmosphere, and when the second port is in a second closed position, the fluid chamber of the handle portion is not vented to the atmosphere;
a shaft extending along a shaft axis from a proximal end to a distal end, wherein one or more inner surfaces of the shaft defines a shaft channel that extends from a shaft channel proximal end at the proximal end of the shaft to a shaft channel distal end at the distal end of the shaft, wherein the shaft is rotatably coupled to the handle portion such that the shaft channel proximal end is disposed within a third portion of the fluid chamber of the handle portion such that the shaft channel is in fluid communication with the fluid chamber of the handle portion; and
a first suction tip removably secured to the distal end of the shaft, the first suction tip having a first cross-sectional shape that is configured to engage a first type of tissue on a patient, wherein the first suction tip comprises a deformable material,
wherein when the first port is coupled to the vacuum source, and when the second port is in the second closed position, the distal end of the shaft is at a low pressure via the vacuum source such that a portion of the first suction tip engages a portion of the first type of tissue of the patient and body fluid at or adjacent to the portion of the first type of tissue of the patient displaces from the shaft channel distal end through the shaft channel, into the fluid chamber, through the first port channel, and out of the open end portion of the first port, and
wherein when the first port is coupled to the vacuum source, and when the second port is in the first open position, the fluid chamber is vented to the atmosphere such that the distal end of the shaft is not at the low pressure of the vacuum source, and body fluid at or adjacent to the portion of the first type of tissue of the patient is not displaced from the shaft channel distal end through the shaft channel.

2. The surgical device of claim 1, further comprising a rotational adapter fixedly secured to a portion of the shaft at or adjacent to the proximal end of the shaft, the rotation adapter comprising:
an insertion portion extending along the shaft axis from a proximal end of the rotational adapter to the distal end of the handle portion, the insertion portion being disposed with a fourth portion of the fluid chamber such that the insertion portion is configured to rotate relative to the handle portion within the fourth portion of the fluid chamber; and
an engagement portion disposed at the distal end of the insertion portion, the engagement portion having a shape of a disk, with a circumferential portion of the disk adapted to be rotated by a user to rotate the rotation adapter and the shaft about the shaft axis.

3. The surgical device of claim 1, wherein the open end portion of the second port is disposed along or adjacent to an outer surface of the handle portion such that the open end portion of the second port is configured to be engaged by a finger of a user holding the handle portion such that:
(a) when the user places the finger completely over the open end portion of the second port, the second port is in the second closed position in which the fluid chamber of the handle portion is not vented to the atmosphere, and
(b) when the user removes the finger from the open end portion of the second port, the second port is in the first open position in which the fluid chamber of the handle portion is vented to the atmosphere.

4. The surgical device of claim 1, wherein at least a portion of the shaft is non-linear.

5. The surgical device of claim 4, wherein the shaft comprises:
a first shaft portion extending from the proximal end of the shaft to a first intermediate point, wherein the first shaft portion extends along a first shaft axis portion of the shaft axis;
a second shaft portion extending from the first intermediate point to a second intermediate point, wherein the second shaft portion extends along a second shaft axis portion of the shaft axis, and wherein the second shaft axis portion is oblique relative to the first shaft axis portion; and
a third shaft portion extending from the second intermediate point to the distal end of the shaft, wherein the third shaft portion extends along a third shaft axis portion of the shaft axis, and wherein the second shaft axis portion is oblique relative to the third shaft axis portion, and the third shaft axis portion is parallel to but radially offset from the first shaft axis portion.

6. The surgical device of claim 1, wherein at least a portion of the handle portion is made from a deformable polymer material.

7. The surgical device of claim 1, further comprising:
a third port disposed on a third portion of the handle portion, the third port defining a third port channel that extends from an open end portion of the third port to a fifth portion of the fluid chamber of the handle portion such that the third port channel is in fluid communication with the fluid chamber of the handle portion, wherein when the third port is in a first open position, the fluid chamber of the handle portion is vented to the atmosphere, and when the third port is in a second closed position and the second port is in the second closed position, the fluid chamber of the handle portion is not vented to the atmosphere.

8. The surgical device of claim 1, wherein the shaft axis is linear from the proximal end of the shaft to the distal end of the shaft.

9. The surgical device of claim 1, wherein the shaft is a molded plastic material.

10. The surgical device of claim 1, wherein the fluid chamber extends within the interior portion of the handle portion from a proximal end to a distal end.

11. A kit comprising:
a surgical device comprising:
a handle portion extending along a handle axis from a distal end to a proximal end, the handle portion having a fluid chamber that extends within an interior portion of the handle portion;
a first port disposed on a first portion of the handle portion, the first port defining a first port channel that extends from an open end portion of the first port to a first portion of the fluid chamber of the handle portion such that the first port channel is in fluid communication with the fluid chamber of the handle portion, wherein the first port is configured to be coupled to a vacuum source;
a second port disposed on a second portion of the handle portion, the second port defining a second port channel that extends from an open end portion of the second port to a second portion of the fluid chamber of the handle portion such that the second port channel is in fluid communication with the fluid chamber of the handle portion, wherein when the second port is in a first open position, the fluid chamber of the handle portion is vented to the atmosphere, and when the second port is in a second closed position, the fluid chamber of the handle portion is not vented to the atmosphere; and
a shaft extending along a shaft axis from a proximal end to a distal end, wherein one or more inner surfaces of the shaft defines a shaft channel that extends from a shaft channel proximal end at the proximal end of the shaft to a shaft channel distal end at the distal end of the shaft, wherein the shaft is rotatably coupled to the handle portion such that the shaft channel proximal end is disposed within a third portion of the fluid chamber of the handle portion such that the shaft channel is in fluid communication with the fluid chamber of the handle portion;
a first suction tip configured to be removably secured to the distal end of the shaft, the first suction tip having a first cross-sectional shape that is configured to engage a first type of tissue on a patient, wherein the first suction tip comprises a first deformable material; and
a second suction tip configured to be removably secured to the distal end of the shaft, the second suction tip having a second cross-sectional shape that is configured to engage a second type of tissue on the patient, wherein the second suction tip comprises a second deformable material,
wherein when the first port is coupled to the vacuum source, and when the second port is in the second closed position, the distal end of the shaft is at a low pressure via the vacuum source such that (a) a portion of the first suction tip engages a portion of the first type of tissue of the patient of (b) a portion of the second suction tip engages a portion of the second type of tissue of the patient, and body fluid at or adjacent to the portion of the first type of tissue or the second type of tissue of the patient displaces from the shaft channel distal end through the shaft channel, into the fluid chamber, through the first port channel, and out of the open end portion of the first port, and
when the first port is coupled to the vacuum source, and when the second port is in the first open position, the fluid chamber is vented to the atmosphere such that the distal end of the shaft is not at the low pressure of the vacuum source, and body fluid at or adjacent to the portion of the first type of tissue of the patient or the second type of tissue of the patient is not displaced from the shaft channel distal end through the shaft channel.

12. The kit of claim 11, wherein the first deformable material is the same as the second deformable material.

13. The kit of claim 11, wherein at least a portion of the shaft is non-linear.

14. The kit of claim 13, wherein the shaft comprises:
a first shaft portion extending from the proximal end of the shaft to a first intermediate point, wherein the first shaft portion extends along a first shaft axis portion of the shaft axis;
a second shaft portion extending from the first intermediate point to a second intermediate point, wherein the second shaft portion extends along a second shaft axis portion of the shaft axis, and wherein the second shaft axis portion is oblique relative to the first shaft axis portion; and
a third shaft portion extending from the second intermediate point to the distal end of the shaft, wherein the third shaft portion extends along a third shaft axis portion of the shaft axis, and wherein the second shaft axis portion is oblique relative to the third shaft axis portion, and the third shaft axis portion is parallel to but radially offset from the first shaft axis portion.

15. The kit of claim 11, the surgical device further comprising a rotational adapter fixedly secured to a portion of the shaft at or adjacent to the proximal end of the shaft, the rotation adapter comprising:
an insertion portion extending along the shaft axis from a proximal end of the rotational adapter to the distal end of the handle portion, the insertion portion being disposed with a fourth portion of the fluid chamber such that the insertion portion is configured to rotate relative to the handle portion within the fourth portion of the fluid chamber; and
an engagement portion disposed at the distal end of the insertion portion, the engagement portion having a shape of a disk, with a circumferential portion of the disk adapted to be rotated by a user to rotate the rotation adapter and the shaft about the shaft axis.

16. The surgical device of claim 11, wherein the open end portion of the second port is disposed along or adjacent to an outer surface of the handle portion such that the open end portion of the second port is configured to be engaged by a finger of a user holding the handle portion such that:
(a) when the user places the finger completely over the open end portion of the second port, the second port is in the second closed position in which the fluid chamber of the handle portion is not vented to the atmosphere, and
(b) when the user removes the finger from the open end portion of the second port, the second port is in the first open position in which the fluid chamber of the handle portion is vented to the atmosphere.

17. The kit of claim 11, further comprising:
a third suction tip configured to be removably secured to the distal end of the shaft, the third suction tip having a third cross-sectional shape that is configured to engage a third type of tissue on the patient, wherein the third suction tip comprises a third deformable material.

18. The kit of claim 10, the surgical device further comprising:
a third port disposed on a third portion of the handle portion, the third port defining a third port channel that extends from an open end portion of the third port to a fifth portion of the fluid chamber of the handle portion such that the third port channel is in fluid communication with the fluid chamber of the handle portion, wherein when the third port is in a first open position, the fluid chamber of the handle portion is vented to the atmosphere, and when the third port is in a second closed position and the second port is in the second closed position, the fluid chamber of the handle portion is not vented to the atmosphere.
